(19) United States
(12) Patent Application Publication
(10) Pub. No.: US 2019/0070034 A1
(43) Pub. Date: Mar. 7, 2019

(54) ADHESIVE FOR MOIST TISSUE AND PERISTOMAL DEVICE MADE USING THE SAME

(71) Applicant: HOLLISTER INCORPORATED, Libertyville, IL (US)

(72) Inventor: Bettakeri S. Udayakumar, Darien, IL (US)

(21) Appl. No.: 15/538,545

(22) PCT Filed: Jan. 28, 2016
(86) PCT No.: PCT/US2016/015406

§ 371 Date: Jun. 21, 2017

Related U.S. Application Data
(60) Provisional application No. 62/108,726, filed on Jan. 28, 2015.

(51) Int. Cl. A61F 5/443; A61L 24/00; A61L 24/04; C08L 83/04; A61L 28/00; C08G 77/20; C08G 77/12

(52) U.S. Cl. CPC A61F 5/443; A61L 24/0094; A61L 24/043; C08L 83/04; A61L 28/0096; C08L 2203/02; A61L 28/0011; A61L 28/0026; C08G 77/20; C08G 77/12; A61L 2400/14; A61L 28/0015

(57) ABSTRACT

An adhesive for moist tissue is formulated including a silicone elastomer, a crosslinked polyacrylic acid polymer and a sodium polyacrylate based superabsorbent polymer. The adhesive adheres well to moist tissue, such as a mucosal skin surface. An ostomy skin barrier including a stoma seal made using the adhesive is also disclosed.

### BACKGROUND

The present disclosure relates to adhesives for moist tissue, and more particularly to peristomal devices made using an adhesive for moist tissue.

An ostomy appliance or system is a medical device or prosthetic that provides a means for collecting waste from a stoma typically created as a result of a surgical procedure to divert a portion of the colon or small intestine. One type of ostomy appliance is a pouch that is attached to a user around the stoma or the peristomal area.

Typically, a skin barrier including an inlet opening to receive a stoma is used to attach an ostomy appliance, such as a pouch, to a user. Leakage of stoma effluent can weaken the seal between a skin barrier and a user's skin, and irritate peristomal skin and cause infection. Peristomal skin irritation and infection can be very difficult to cure. Thus, efforts have been made to provide a skin barrier that can fit and seal around a stoma outer wall to reduce stoma effluent leakage. However, stoma effluent leakage remains as a serious problem for ostomates.

Thus, any improvements to reduce the risk of stoma effluent reaching peristomal skin are of great importance to ostomates. The present disclosure provides an adhesive composition for moist tissue that can seal around a stoma, and an improved skin barrier according to various embodiments to reduce the risk of stoma effluent reaching peristomal skin.

### BRIEF SUMMARY

Adhesive compositions for moist tissue may be formulated with a silicone elastomer, a crosslinked polyacrylic acid polymer, and a sodium polyacrylate based superabsorbent polymer according to various embodiments. The adhesive compositions are configured to adhere to moist tissue, such as the mucosal wall of a stoma, mucocutaneous base of stoma, and partially or completely denuded skin. Skin barriers for ostomy appliances including a stoma seal formed using such an adhesive composition are disclosed according to various embodiments. The stoma seal is configured to hug a stoma and adhere to the base and outer walls of the stoma, such that the stoma seal may accommodate peristalsis or stomal movement during use.

The adhesive compositions for moist tissue may also be used as a skin adhesive for attaching various devices including medical devices. For example, the adhesive can be used for incontinence products, such external male catheters. The adhesive compositions for moist tissue may also be used in wound care devices.

In one aspect, an adhesive composition for moist tissue formulated with about 80 weight percent (wt. %) to about 98 wt. % of a two-part addition curing silicone composition and about 2 wt. % to about 20 wt. % of at least one hydrophilic component is provided according to various embodiments. When cured, adhesive composition forms a viscoelastic adhesive that adheres to a moist mucocutaneous tissue and a mucosal tissue.

In some embodiments, the hydrophilic component may include a crosslinked polyacrylic acid polymer and/or a sodium polyacrylate based superabsorbent polymer. The two-part addition curing silicone composition may comprise a component A including a platinum catalyst and vinyl functional polymers (R—CH═CH₂) and a component B comprising silicone hydride groups (—SiH).

In an embodiment, the viscoelastic adhesive may have a total work adhesion greater than about 7 N·mm, and an elongation before detaching greater than about 3.0 mm and less than about 150 mm when tested according the Spherical Probe Tack and Adhesion test method described in Examples and Test Results section of the present disclosure. Further, the viscoelastic adhesive may have a total work adhesion greater than about 100 g·mm, and an elongation before detaching greater than about 5.0 mm and less than about 50 mm when tested according the Moist Tissue Tack and Adhesion test method described in Examples and Test Results section of the present disclosure. Further, the viscoelastic adhesive may have saline solution absorption over 40 days of about 7 wt. % to about 80 wt. % when tested according to the Absorption Test in 0.9% NaCl solution in Examples and Test Results section of the present disclosure.

In an embodiment, the adhesive for moist tissue may be formed into a stoma seal having a ring-like shape body, which may maintain the shape and structure of the body after being soaked in a 0.9% NaCl solution for 40 days.

In another aspect, an ostomy skin barrier including a faceplate with a first inlet opening defined therein, a stoma seal, a first adhesive layer, and a second adhesive layer is provided according to various embodiments. The stoma seal may be provided in the first inlet opening. The stoma seal may have a ring-like shaped body with a second inlet opening configured to receive a stoma defined therein. The first adhesive layer may be provided on the faceplate, and the second adhesive layer may be provided on the faceplate surrounding the stoma seal. Each of the stoma seal, the first adhesive, and the second adhesive may be formed from a different adhesive formulation.

In some embodiments, the first adhesive layer may be formed from a hydrophilic adhesive and the second adhesive layer is formed from a hydrophobic adhesive. For example, the first adhesive layer may be formed from a hydrocolloid adhesive or an acrylic adhesive, and the second adhesive layer may be formed from a silicone adhesive. Further, the second adhesive layer may be arranged between the stoma seal and the first adhesive layer. In such an embodiment, the hydrophobic second adhesive layer may function as a barrier between the stoma seal and the first adhesive layer to minimize a risk of any stoma effluent leak around the stoma sleeve reaching the first adhesive layer. The stoma seal may be formed from an adhesive composition for moist tissue prepared according to any of the foregoing embodiments.

In an embodiment, the second adhesive layer may have a ring-like shape and may be arranged on the first adhesive layer, such that an outer peripheral portion of the first adhesive layer remains exposed for attachment to a user. The first inlet opening may be defined by inner peripheries of the faceplate, the first adhesive layer, and the second adhesive layer, in which the stoma seal may be provided. The stoma seal may have a thickness equal to or greater than a combined thickness of the faceplate, the first adhesive layer, and the second adhesive layer, such that the stoma seal may extend longitudinally from a pouch side inner periphery of the faceplate to a body side inner periphery of the second adhesive layer. A cover may be provided on a body side surface of the stoma seal, and a sealing layer may be provided on a pouch side surface of the stoma seal, in which the pouch side surface of the stoma seal may be secured to the sealing layer.

Further, the ostomy skin barrier may include a first release liner provided on the exposed outer peripheral portion of the first adhesive layer, and a second release liner provided on a body side surface the second adhesive layer, in which the stoma seal may have a thickness equal to or greater than a combined thickness of the faceplate, the first adhesive layer, the second adhesive layer, and the second release liner, such that the stoma seal extends longitudinally from a pouch side inner periphery of the faceplate to a body side inner periphery of the second release liner.

In an embodiment, the first adhesive may be formed from an acrylic adhesive, and the second adhesive layer may be formed from a hydrocolloid adhesive, and the sealing layer may be formed from a silicone. The stoma seal may be formed from an adhesive composition for moist tissue prepared according to any of the foregoing embodiments. Further, the ostomy skin barrier may include a body side coupling ring attached on a pouch side surface of the faceplate, in which the sealing layer is provided over the stoma seal and an inner peripheral portion of the faceplate inside an inner perimeter of the body side coupling ring.

In another aspect, an ostomy skin barrier comprising a faceplate, a first adhesive layer, a backing layer, a second adhesive layer, and a stoma seal is provided. The faceplate may include an opening defined by an inner periphery of the faceplate, and the first adhesive layer may be provided on a body side surface of the faceplate, in which an inner periphery of the first adhesive layer may substantially line up with the inner periphery of the faceplate. The backing layer may have a ring-like shape and may be provided on an inner peripheral portion of the first adhesive, such that an outer peripheral portion of the first adhesive may be exposed for attachment to a user. Further, an inner diameter of the backing layer may be less than inner diameters of the faceplate and the first adhesive layer, such that an inner peripheral portion of the backing layer may extend beyond the inner peripheries of the faceplate and the first adhesive layer. The second adhesive layer having a ring-like shape may be provided on an outer peripheral portion of the backing layer, such that the outer peripheral portion of the backing layer may be secured between the first adhesive layer and the second adhesive layer, in which the second adhesive layer includes an opening defined by an inner periphery of the second adhesive. The stoma seal may be provided in the opening of the second adhesive layer and attached to an inner peripheral portion of the backing layer. The stoma seal may have a ring-like shape body including an inlet opening configured to receive a stoma.

In an embodiment, each of the stoma seal, the first adhesive layer, and the second adhesive layer may be formed from a different adhesive formulation. For example, the first adhesive layer may be formed from an acrylic adhesive, and the second adhesive layer may be formed from a hydrocolloid adhesive, and the stoma seal may be formed from a silicone adhesive composition for moist tissue. The backing layer may be formed from a polymeric film having a thickness of about 1 mil to about 7 mil.

In some embodiments, the stoma seal may be formed from an adhesive composition for moist tissue prepared according to any of the foregoing embodiments. The backing layer may be formed from a thermoplastic urethane-phenoxy film having a thickness of about 5 mil. Further, the ostomy skin barrier may include a first release liner provided on the exposed outer peripheral portion of the first adhesive layer, and a second release liner provided on a body side surface of the second adhesive layer, and a cover provided on a body side surface of the stoma seal.

Other aspects, objectives and advantages will become more apparent from the following detailed description when taken in conjunction with the accompanying drawings.

### BRIEF DESCRIPTION OF THE DRAWINGS

The benefits and advantages of the present embodiments will become more readily apparent to those of ordinary skill in the relevant art after reviewing the following detailed description and accompanying drawings, wherein:

FIG. 1 is a perspective view of a skin barrier including a stoma seal according to a first embodiment;

FIG. 2 is a perspective top view (body side view) of the skin barrier of FIG. 1;

FIG. 3 is a cross sectional view of the skin barrier of FIG. 1 taken along line A-A;

FIG. 4 is a perspective view of an ostomy pouch configured to engage with the skin barrier of FIG. 1 according to an embodiment;

FIG. 5 is a perspective view of a stoma sleeve according to an embodiment;

FIG. 6 is a perspective view of a skin barrier including a stoma seal according to a second embodiment;

FIG. 7 is a perspective top view (body side view) of the skin barrier of FIG. 6;

FIG. 8 is a perspective bottom view (pouch side view) of the skin barrier of FIG. 6;

FIG. 9 is an exploded view of the skin barrier of FIG. 6;

FIG. 10 is a cross sectional view of the skin barrier of FIG. 6 taken along line A-A;

FIG. 11 is a cross sectional view of a skin barrier including a stoma seal according to a third embodiment;

FIG. 12 is a photograph of a moist tissue adhesive sample adhering to a ball probe during a Spherical Probe Tack and Adhesion test run;

FIGS. 13A-D are photographs taken during a Moist Tissue Tack and Adhesion test run;

FIGS. 14 A-B are photographs of a moist tissue adhesive sample attached and stretched from a porcine epidermis in buffer solution at t=0 and t=16 hours, respectively;

FIGS. 15A-D are photographs of a moist tissue adhesive sample attached to a moist porcine epidermis after being soaked in buffer solution for 5 days, which was subsequently detached from the moist porcine epidermis cleanly;

FIG. 16 is a graph of 0.9% NaCl solution absorption rate of moist tissue adhesive samples;

FIG. 17 is a graph of 0.9% NaCl solution absorption rate of a moist tissue adhesive sample; and

FIG. 18 is a bar graph showing 0.9% NaCl solution absorbance of hydrocolloid samples and a moist tissue adhesive sample.

### DETAILED DESCRIPTION

While the present disclosure is susceptible of embodiment in various forms, there is shown in the drawings and will hereinafter be described presently preferred embodiments with the understanding that the present disclosure is to be considered an exemplification and is not intended to limit the disclosure to the specific embodiments illustrated.

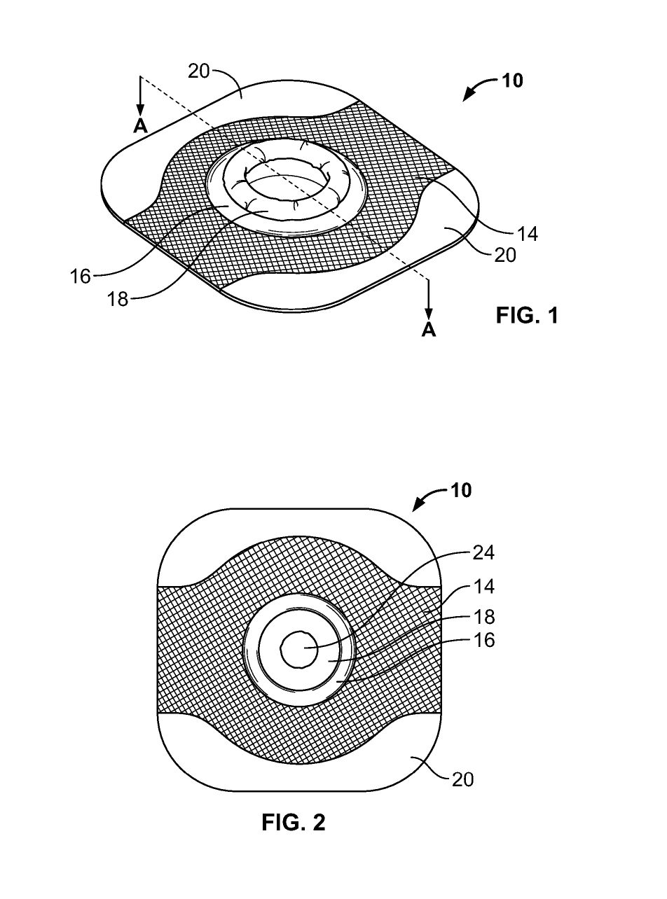

Referring to FIGS. 1-3, an embodiment of a skin barrier 10 for an ostomy appliance is shown. The skin barrier 10 generally includes a faceplate 12, a first adhesive layer 14, a second adhesive layer 16, a stoma seal 18, a release liner 20 and inlet opening 24 for receiving a stoma 26. The skin barrier 10 may also include a body side coupling ring 22 for attaching an ostomy appliance, such as a pouch 30 shown in FIG. 4. The body side coupling ring 22 is configured to mate with a pouch side coupling ring 32 of the pouch 30, such that the pouch 30 may be mechanically secured to the skin barrier 10 when the coupling rings 22, 32 are engaged together.

In use, the skin barrier 10 is attached to a user, such that a stoma is received through the inlet opening 24, and the first adhesive layer 14 and the second adhesive layer 16 are attached to peristomal skin 28 surrounding the stoma 26. The release liner 20 is removed before the first adhesive layer 14 is attached to peristomal skin 28. The stoma seal 18 adheres to the base of the stoma 29 and hugs the stoma 26 to seal around the outer walls of the stoma 25 and moves with the stoma 26 during use. After the skin barrier 10 is attached to the user, the pouch 30 may be attached to the skin barrier 10 by engaging the coupling rings 22, 32 together.

The faceplate 12 may be formed from a gas-permeable, water-resistant microporous material. Preferably, the faceplate 12 is highly flexible, so that it will conform readily to body contours and body movements, and relatively strong and durable. The first adhesive layer 14 may be formed from a hydrophilic adhesive, while the second adhesive layer 16 may be formed from a hydrophobic adhesive. The stoma seal 18 may be formed from an adhesive composition for moist tissue.

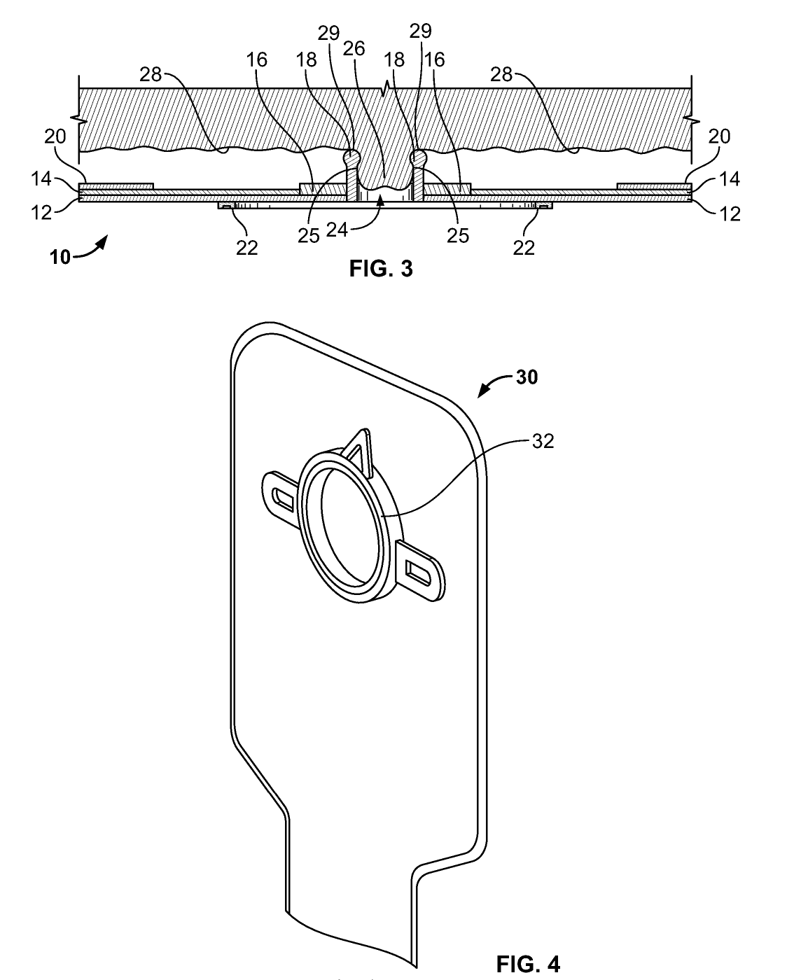

As illustrated in FIG. 3, the stoma seal 18 has a sleeve like shape including a generally cylindrical body with an inlet opening 24 defined therein, such that a stoma 26 may be received through the inlet opening 24. The stoma seal 18 is configured to seal at the base 29 and around the outer walls 25 of the stoma 26 and move with the stoma during use to maintain the seal around the stoma 26 to isolate peristomal area from stoma effluents. The second adhesive layer 16 is provided surrounding the stoma seal 18. The second adhesive layer 16 functions as a barrier between the stoma seal 18 and first adhesive layer 14 to minimize a risk of any stoma effluent that may leak around the stoma seal 18 from reaching the first adhesive layer 14 and being absorbed by the first adhesive layer 14. Stoma effluent can cause peristomal skin irritation. Thus, it is highly desirable to minimize stoma effluent absorption by the first adhesive skin layer 14, which is in direct contact with a relatively wide peristomal skin area. The second adhesive layer 16 may be formed from a hydrophobic adhesive, such as a silicone adhesive. As such, the second adhesive layer 16 does not absorb stoma effluent. Further, the second adhesive layer 16 may redirect any stoma effluent leakage towards the stoma seal 18 and prevent the stoma effluent from reaching the first adhesive layer 14.

The first adhesive layer 14 may be formed of a suitable pliable and tacky barrier material capable of engaging and sealing the peristomal area. Such barrier materials are well known in the art. For example, the first adhesive may be formed of a medical-grade pressure sensitive adhesive that can adhesively secure the skin barrier 10 to a patient's skin in the peristomal region. Preferably, the first adhesive is formed from a hydrophilic adhesive, such as a hydrocolloid adhesive composition or an acrylic adhesive.

As shown in FIG. 3, a release liner 20 may be provided to cover a portion of the first adhesive layer 14 for easy handing and positioning of the skin barrier 10. The release liner 20 may be removed by a user before the first adhesive layer 14 is attached to peristomal skin 28. Although not shown in FIGS. 1-3, additional release liners or a release cover may be provided over the second adhesive layer 16 and/or the stoma seal 18.

The stoma seal 18 may be formed from an adhesive composition for moist tissue comprising a hydrophilic dispersion in a hydrophobic matrix. The adhesive composition may be formulated to have good adhesion to moist mucocutaneous area of a stoma base 29 and wet mucosal stoma walls 25 and remain flexible during use, such that a stoma seal formed from the adhesive composition may bond and seal around outer walls and base of a stoma and move with the stoma during use. Further, the adhesive composition may be formulated to detach cleanly from a stoma without leaving residues after use. In some embodiments, the stoma seal 18 may be formed from an adhesive composition comprising a hydrophilic component, such as polyacrylic acid, dispersed in a hydrophobic matrix, such as silicone adhesive. The structure of such an adhesive composition is supported by the hydrophobic matrix, while the hydrophilic component absorbs water and/or stoma effluent.

In an embodiment, an adhesive composition for moist tissue may be formulated with about 70 weight percent (wt. %) to about 99 wt. % of silicone adhesive and about 1 wt. % to about 30 wt. % of hydrophilic components, preferably about 80 wt. % to about 98 wt. % of silicone adhesive and about 2 wt. % to about 20 wt. % of hydrophilic components, more preferably, about 85 wt. % to about 97 wt. % of silicone adhesive and about 3 wt. % to about 15 wt. % of hydrophilic components. In some embodiments, the adhesive composition may also include about 0.05 wt. % to about 5 wt. % of fibers, preferably 0.1 wt. % to about 2 wt. % of fibers, more preferably 0.5 wt. % to about 1 wt. % of fibers. Suitable fibers include fibrillated high density polyethylene (HDPE) fibers having an average diameter of about 5 μm and other similar fibers and fillers. The adhesive composition may also include about 0.05 wt. % to about 1 wt. % of ceramide, preferably about 0.1 wt. % to about 0.5 wt. % of ceramide.

Suitable silicone adhesives for adhesive compositions for moist tissue include two-part addition curing silicone compositions that cure at room temperature, which may also be referred to as RTV-2 silicone herein. An example of suitable RTV-2 silicones is a two-part platinum (Pt) catalyzed silicone gel elastomer composition including component A comprising Pt and unsaturated polymers with a vinyl group, such as R—CH═CH₂, and component B comprising silicone reactive groups, such as R′—SiH, which participates in Pt catalyzed addition reaction, known as hydrosilylation.

Suitable hydrophilic components include polyacrylic acid and superabsorbent polymers, such as sodium polyacrylate, cross linked cellulose polymers, and pectin.

In an embodiment, an adhesive composition for moist tissue may comprise a two-part Pt catalyzed silicone elastomer composition including component A comprising Pt and vinyl functional polymers (R—CH═CH₂) and component B comprising silicone hydride groups (—SiH), and hydrophilic components. The adhesive composition may be formulated with a sufficient quantity of the two-part Pt catalyzed silicone elastomer to hold the shape and structure after the adhesive composition is molded and cured into a stoma seal. Further, the adhesive composition may be formulated with sufficient quantities of hydrophilic components, such that the stoma seal may adhere to outer walls and mucocutaneous area of a stoma to seal around the stoma and move along with the stoma during use, while absorbing stoma effluent that may leak around the stoma.

In an embodiment, an adhesive composition for moist tissue may comprise about 85 wt. % to about 97 wt. % of a two-part Pt catalyzed silicone elastomer and about 3 wt. % to about 15 wt. % of hydrophilic components, wherein the hydrophilic components may comprise about 1 wt. % to about 14 wt. % of a crosslinked polyacrylic acid polymer and about 1 wt. % to about 14 wt. % of a sodium polyacrylate based superabsorbent polymer, preferably about 3 wt. % to about 14 wt. % of a crosslinked polyacrylic acid polymer, and about 1 wt. % to about 12 wt. % of a sodium polyacrylate based superabsorbent polymer.

When cured, an adhesive composition for moist tissue may form a stoma seal that adheres to moist mucocutaneous area of a stoma base and wet mucosal stoma walls to seal around a stoma. A cured adhesive composition for moist tissue may have a total work adhesion greater than 5 N·mm, preferably greater than 7 N·mm, more preferably greater than 10 N·mm when tested according to the Spherical Probe Tack and Adhesion Test described in Examples and Test Results section of the present disclosure. Further, the cured adhesive composition may have an elongation before detaching greater than 1 mm, preferably greater than 3.0 mm and less than 150 mm, and more preferably greater than 5.0 mm and less than 50 mm when tested according to the Spherical Probe Tack and Adhesion Test described in Examples and Test Results section of the present disclosure. Good elastic properties of the adhesive, which may be tested by the elongation test during which the adhesive is stretched without losing contact with the spherical probe, may indicate that a stoma sleeve formed from such an adhesive may stretch and contract with a stoma as the stoma moves with peristaltic motions and bending and stretching of user's body.

Further, the cured adhesive composition may have a total work adhesion greater than 100 g·mm, preferably greater than 200 g·mm, more preferably greater than 400 g·mm when tested according to the Moist Tissue Tack and Adhesion Test described in Examples and Test Results section of the present disclosure. The cured adhesive composition may also have an elongation before detaching greater than 1 mm, preferably greater than 5.0 mm and less than 50 mm, and more preferably greater than 7.0 mm and less than 30 mm when tested according to the Moist Tissue Tack and Adhesion Test described in Examples and Test Results section of the present disclosure.

Further, the cured adhesive composition may have a saline solution absorption of about 5 wt. % to about 100 wt. % over 40 days, preferably about 7 wt. % to about 80 wt. % over 40 days, and more preferably about 10 wt. % to about 60 wt. % over 40 days when tested according to the Absorption Test in 0.9% NaCl solution in Example and Test Results section of the present disclosure.

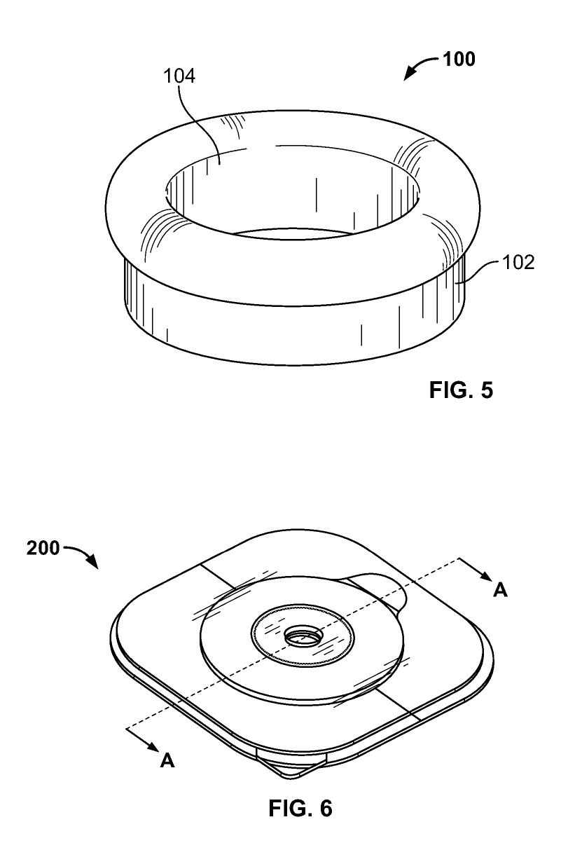

In another embodiment, the adhesive composition for moist tissue formulated according to various embodiments of the present disclosure may be used to make a stoma sleeve 100 shown in FIG. 5. The stoma sleeve 100 may have a generally cylindrical shape body 102 with an inlet opening 104 defined therein for receiving a stoma. The stoma sleeve 100 may be sold as an ostomy accessory that may be used with commercially available ostomy skin barriers. In yet another embodiment, a one-piece ostomy pouch may include a skin barrier including a stoma seal that is similarly configured as the stoma seal 18 of FIGS. 1-3 or a stoma sleeve 100 of FIG. 5.

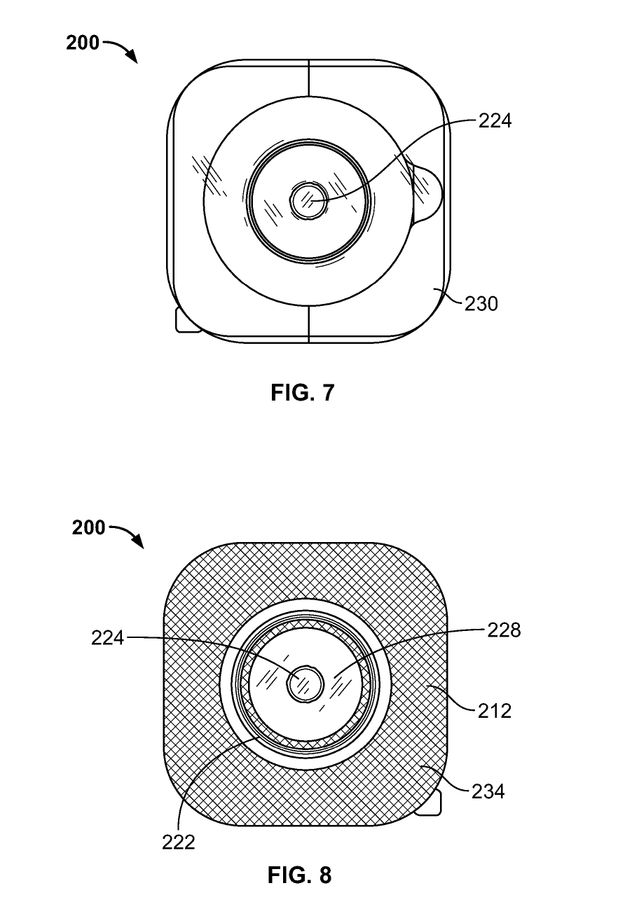

FIGS. 6-10 illustrate a skin barrier 200 according to a second embodiment. The skin barrier 200 may generally include a faceplate 212, a first adhesive layer 214, a second adhesive layer 216, a stoma seal 218, first and second release liners 220, 226, a backing layer 228, and a cover tray 230. The skin barrier 200 also may also include an inlet opening 224 for receiving a stoma, and a body side coupling ring 222 for engaging a pouch side coupling ring 32 (FIG. 4) to attach an ostomy pouch. The inlet opening 224 may be defined by an opening 202 defined by an inner periphery of the stoma seal 218 and an opening 204 defined by an inner periphery of the backing layer 228. The opening 202 and opening 204 may have the same generally circular shape and approximately the same diameter.

The faceplate 212 and the first adhesive 214 may include an opening 206 defined by inner peripheries of the faceplate 212 and first adhesive 214. The second adhesive 216 also includes an opening 208 defined by an inner periphery. The opening 206 and opening 208 may have the same generally circular shape and approximately the same diameter. The diameter of the opening 206 and opening 208 may be greater than the opening 202 and opening 204.

The faceplate 212 may be formed using any of the suitable gas-permeable, water-resistant microporous materials described above with regard to the first embodiment faceplate 12. For example, the faceplate 214 may be formed from a single layer of a nonwoven material or a multiplayer material including a polymeric film and/or nonwoven. The body side coupling ring 222 may be attached to the faceplate 212 on a pouch side surface 235.

The first adhesive layer 214 may be provided on a body side surface 236 of the faceplate 212. The first adhesive layer 214 may be formed from a suitable pliable tacky barrier material having a good adhesion to user's peristomal skin. For example, the first adhesive 214 may be formed from a medical-grade pressure sensitive adhesive, such as an acrylic adhesive. The first adhesive layer 214 may be coated or laminated on the faceplate 212. In an embodiment, the first adhesive layer 214 may be provided on the entire body side surface 236 of the faceplate 212. In other embodiments, the first adhesive layer 214 may be provided on an outer portion less than the entire body side surface 236 of the faceplate 212. In the embodiment of FIGS. 6-10, the first adhesive layer 214 covers the entire body side surface 236 of the faceplate 212, and the opening 206 is defined by generally circular inner peripheries of the faceplate 212 and the first adhesive layer 214.

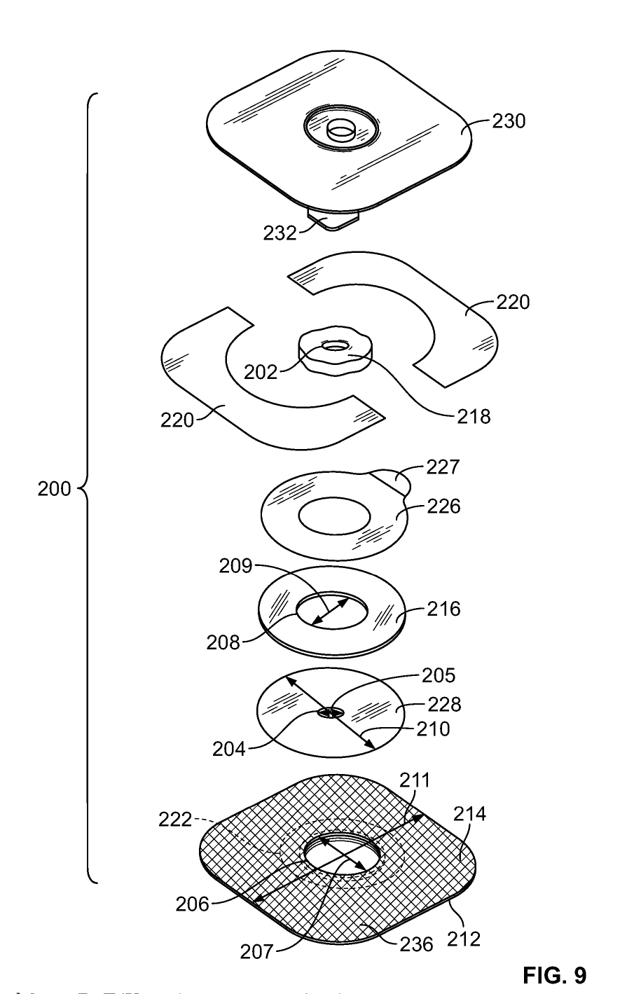

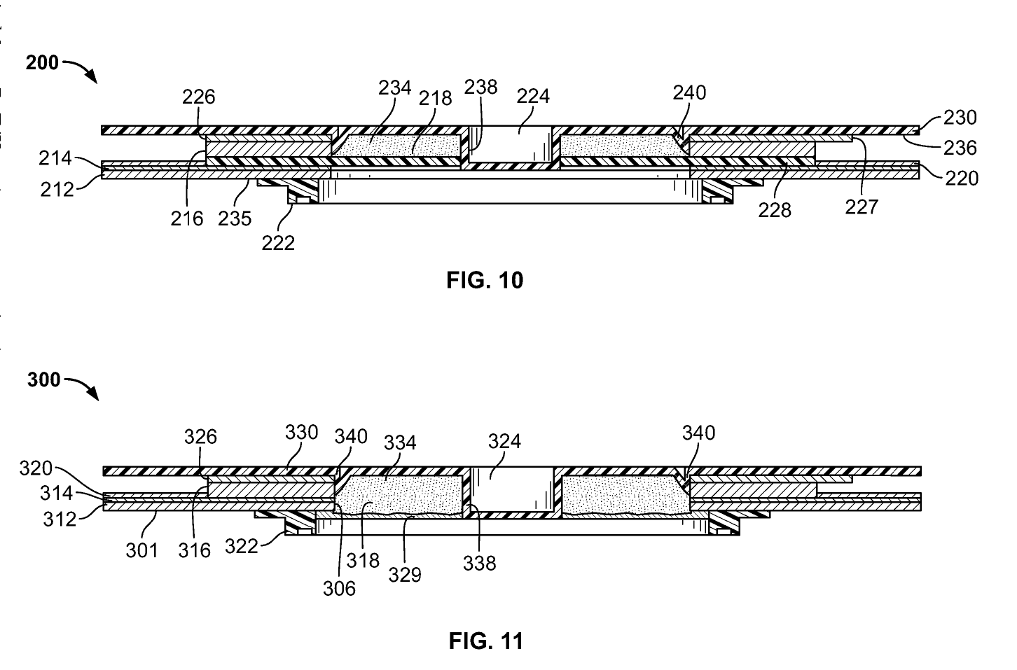

The backing layer 228 may be provided on the first adhesive layer 214, such that an outer peripheral portion of the backing layer 228 is sandwiched between the first adhesive 214 and the second adhesive 216. The backing layer 228 may have a generally ring-like shape including a generally circular inner periphery defining the opening 204. A diameter 210 of the backing layer 228 may be greater than a diameter 207 of the opening 206 and less than a width 211 of the faceplate 212, such that the backing layer 228 may cover an inner peripheral portion of the first adhesive layer 214 and leave an outer peripheral portion of the first adhesive layer 214 exposed for attachment to a user. The exposed outer peripheral portion may be covered with the first release liner 220, which may be removed prior to attachment to a user. The first release liner 220 may be provided as a single piece or multiple pieces. For example the first release liner 220 may comprise two release liner pieces as shown in FIG. 9. Further, the opening 204 may have a diameter 205, which is smaller than that of the opening 206, such that the backing layer 228 extends beyond the inner peripheries of the faceplate 212 and the first adhesive layer 214 as shown in FIG. 10.

The backing layer 228 may be formed from a suitable thin polymeric film, which may be sufficiently flexible to move with the stoma seal 218. For example, the backing layer 228 may be formed from a thermoplastic urethane-phenoxy film having a thickness of about 1 mil to about 7 mil, preferably about 2 mil to about 6 mil, and more preferably about 3 mil to about 5 mil.

The second adhesive layer 216 may be provided on an outer peripheral portion of the backing layer 228, such that the outer peripheral portion of the backing layer 228 is secured between the first adhesive layer 214 and the second adhesive layer 216 as shown in FIG. 10. The second adhesive layer 216 may be formed of a suitable pliable and tacky barrier material capable of engaging and sealing the peristomal area. For example, the second adhesive layer 216 may be formed from a hydrophilic medical-grade adhesive composition, such as a hydrocolloid adhesive composition. In some embodiment, the first adhesive layer 214 and the second adhesive layer 216 may be formed from the same adhesive composition.

The second adhesive layer 216 may have a generally ring-like shape including the opening 208 defined by a generally circular inner periphery. The opening 208 may have a diameter 209, which is larger than the diameter 205 of the opening 204 in the backing layer 228, such that an inner peripheral portion of the backing layer 228 is not covered by the second adhesive layer 216. In an embodiment, the diameter 209 of the opening 208 in the second adhesive layer 216 may be approximately the same as the diameter 207 of the opening 206 in the faceplate 212, and the outer diameter of the second adhesive layer 216 may be approximately same as that of the backing layer 228.

In some embodiments, the outer diameter of the second adhesive layer 216 may be larger than that of the backing layer 228, such that an outer peripheral portion of the second adhesive layer 216 may be in direct contact with the first adhesive layer 214. The exposed surface of the second adhesive layer 216 may be covered with the second release liner 226, which may be removed prior to attachment to a user. The second release liner 226 may include a tab 227 to facilitate removal.

The stoma seal 218 may be provided on an inner peripheral portion of the backing layer 228. The stoma seal 218 may be formed from a suitable material having sufficient adhesion to a mucosal wall and mucocutaneous base of a stoma. For example, the stoma seal 218 may be formed from an adhesive composition for moist tissue formulated according to various embodiments of the present disclosure. The exposed surface of the stoma seal 218 may be covered with a release liner.

In the embodiment of FIGS. 6-10, the cover tray 230 is provided over the stoma seal 218. The cover tray 230 may be formed from a suitable polymeric material, such as polyethylene terephthalate (PETG), and may have approximately the same outer shape as that of the faceplate 212. The cover tray 230 may be provided with a tab 232 to facilitate removal. The cover tray 230 may include a well 234 defined on a pouch side surface 236 between a cylinder-like center protrusion 238 and a generally circular outer protrusion 240. The cylinder-like center protrusion 238 may be configured such that it may fit snugly in the opening 204 of the backing layer 228 with the inner periphery of the backing layer 228 abutting the cylinder-like center protrusion 238 as shown in FIG. 10. The generally circular outer protrusion 240 may be configured such that it may fit in the opening 208 of the second adhesive layer 216 with the outer wall of the outer protrusion 240 abutting the inner periphery of the second adhesive layer 216. At least the well 234 including the cylinder-like center protrusion 238 and the generally circular outer protrusion 240 may be coated with a release agent, such that the cover tray 230 may be removed prior to attachment to a user.

In some embodiments, the stoma seal 218 may be molded in the cover tray 230 using an adhesive composition for moist tissue. In such embodiments, a pre-cured adhesive composition for moist tissue may be poured into the well 234 and cured to form the stoma seal 218. The cover tray 230 including the stoma seal 218 may be assembled with the rest of skin barrier 200, such that the generally circular outer protrusion 240 may be inserted into the opening 208 of a second adhesive 216 and the cylinder-like center protrusion 238 may be received in the opening 204 in the backing layer 228 as shown in FIG. 10. When assembled, the stoma seal 218 is secured to the inner peripheral portion of the backing layer 228.

FIG. 11 is a cross sectional view of a skin barrier 300 according to a third embodiment. The skin barrier 300 is similarly constructed as the skin barrier 200, and may include a faceplate 312, a first adhesive layer 314, a second adhesive layer 316, a stoma seal 318, first and second release liners 320, 326, and a cover tray 330. The skin barrier 300 also may include an inlet opening 324 for receiving a stoma, and a body side coupling ring 322 for engaging a pouch side coupling ring to attach an ostomy pouch. Unlike the skin barrier 200, the skin barrier 300 does not include a backing layer 228. Instead, the skin barrier 300 may include a sealing layer 329.

In an embodiment, the cover tray 330 is arranged over the second adhesive layer 316 and the second release liner 326, such that a generally circular outer protrusion 340 abuts at least a portion of inner peripheries of second adhesive layer 316 and the second release liner 326. The stoma seal 318 may be arranged in a well 334 defined between the generally circular outer protrusion 340 and a cylinder-like center protrusion 338. The seal layer 329 may be provided on a pouch side surface 301 of the skin barrier 300 over the stoma seal 318 and an inner peripheral portion of the faceplate 312 inside the body side coupling ring 322 as shown in FIG. 11.

The seal layer 329 may be formed from a suitable sealing material. Suitable sealing materials include silicone, such as a RTV-2 silicone composition that cures to form a nontacky silicone layer. Such a RTV-2 silicone composition may have a sufficiently low viscosity, such that the RTV-2 silicone composition may flow on a surface of the stoma seal 318 and faceplate 312 and may cover the corners of the body side coupling ring 322.

In the embodiment of FIG. 11, the faceplate 312 may be provided with the first adhesive layer 314 on the body side surface, and the body side coupling ring 322 may be attached to the pouch side surface. The second adhesive layer 316 having a ring-like shape may be provided on the first adhesive layer 314, such that inner peripheries of the faceplate 312, first adhesive layer 314, and second adhesive layer 316 generally line up to define an opening 306. An outer diameter of the second adhesive layer 316 may be smaller than a width of the faceplate 312, such that an outer peripheral portion of the first adhesive layer 314 is exposed for attachment to a user. The exposed outer peripheral portion of the first adhesive layer 314 may be covered with the first release liner 320. The second adhesive layer 316 may be covered with the second release liner 326 having generally the same shape as the second adhesive layer 316.

The stoma seal 318 may be molded in the cover tray 330 using an adhesive composition for moist tissue. In an embodiment, a pre-cured adhesive composition for moist tissue may be poured into the well 334. The cover tray 330 including the adhesive composition may be assembled with the rest of skin barrier 300, such that the generally circular outer protrusion 340 is inserted into an opening defined by the inner periphery of the second adhesive 216 as shown in FIG. 11. Additional pre-cured adhesive composition may be poured into the well 334 to fill the well up to the pouch side surface 301 of the faceplate 312. When cured, the stoma seal 318 may fill the well 334, and attached to the inner peripheries of the faceplate 312 and first adhesive layer 314 and the exposed inner periphery of the second adhesive layer 316. In some embodiments, the stoma seal 318 may be over molded to cover an outer peripheral edge of the faceplate 312.

In another embodiment, the cover tray 330 may be arranged over the second adhesive layer 316 before pouring a pre-cured adhesive composition. Subsequently, the pre-cured adhesive composition may be poured to fill the well 334 from the pouch side surface 301 and cured to form the stoma seal 318.

A pre-cured low viscosity RTV-2 silicone composition may be poured on the inner perimeter of the body side coupling ring 322 to cover the exposed surface of the stoma seal 318 and an inner peripheral portion of the faceplate 312. When cured, the sealing layer 329 is formed to secure the stoma seal 318.

### Examples and Test Results

Silicone Adhesive mixing procedure: two-part Pt catalyzed silicone compositions (RTV-2 silicone) including component A comprising Pt and vinyl functional polymers (R—CH═CH₂) and component B comprising silicone hydride groups (—SiH) were used to prepare experiment samples. 1:1 ratio of component A and component B was used for all samples. All ingredients were weighed into ajar and mixed well with a mechanical stirrer. They are then poured onto desired trays (design depending on the test performed), degassed under vacuum and placed in a convection oven or on an infrared-belt to expedite the curing process. Some of the silicone compositions cured at room temperature.

### Spherical Probe Tack and Adhesion Test on TA-XT Plus Texture Analyzer

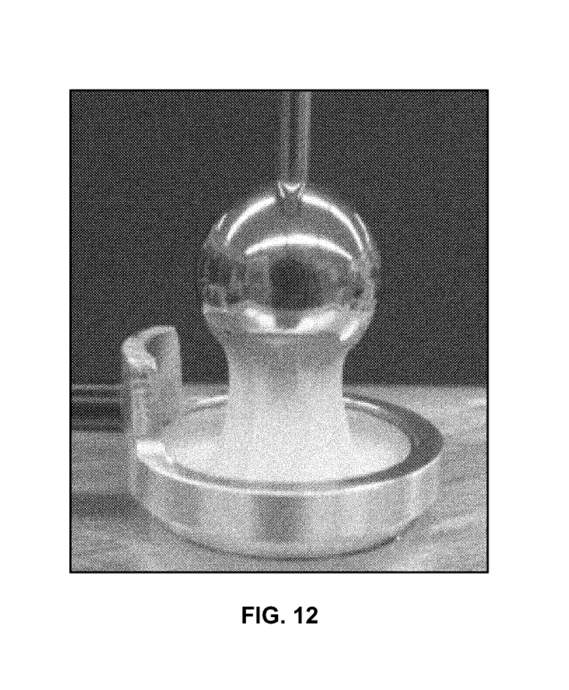

Three grams of each of sample adhesive compositions for moist tissue was placed in a polycarbonate petri dish and degassed to remove trapped air bubbles prior to curing. The adhesive composition samples were allowed to stand at room temperature for a minimum 24 hours prior to testing. A stainless steel spherical ball probe having a diameter of 1.00 inch was used for the test. FIG. 12 is a photograph of a sample adhesive adhering to the ball probe and stretching during a test run. The test parameters included: test speed—0.50 mm/sec, applied force—4.50 Newton, contact time—0.01 sec, trigger type—Auto, trigger force—049 N, test speed—0.50 mm/sec. Test results are summarized in Table 1.

TABLE 1
Spherical Probe Tack and Adhesion Test Results

| | | | crosslinked polyacrylic | | Average Work | Average sample |
|---|---|---|---|---|---|---|
| | | RTV-2 | acid polymer + | | of Adhesion | elongation |
| | RTV-2 | silicone type | sodium polyacrylate | Average | (Area under | before detaching |
| Sample | silicone | (n - sample | based superabsorbent | Peak tack | the curve) | cleanly from the |
| # | (wt. %) | size) | polymer (wt. %) | (N) | (N · mm) | probe (mm) |
| 1 | 90.5 | Type 1 (n = 6) | 9.50 | 2.11 | 23.09 | 15.4 |
| 2 | 98.0 | Type 1 (n = 6) | 2.00 | 1.44 | 4.30 | 4.5 |
| 3 | 92.5 | Type 1 (n = 6) | 7.50 | 1.43 | 11.56 | 9.6 |
| 4 | 91.61 | Type 1 (n = 6) | 8.39 | 2.34 | 33.98 | 25.0 |
| 5 | 90.5 | Type 2 (n = 6) | 9.50 | 1.94 | 37.23 | 25.4 |
| 6¹ | 88.0 | Type 1 (n = 6) | 11.00 | 1.89 | 8.29 | 7.0 |
| 7² | 91.41 | Type 1 (n = 6) | 8.39 | 2.33 | 0.30 | >150³ |
| 8 | 90.5 | Type 3 (n = 6) | 9.50 | 3.19 | 34.27 | 19.6 |
| 9 | 100.0 | Type 4 (n = 6) | 0.00 | 0.04 | 0 | 0.1 |
| 10 | 90.5 | Type 5 (n = 6) | 9.50 | 1.56 | 9.7 | 8.4 |

¹This run also included 1% Fibrillated HDPE fiber (5 μm diameter).
²This run also included 0.2% of pentaerythritol allyl ether as a chain stopper. Although the elongation data appears as zero, in fact, the sample never got fully detached from the spherical probe at the completion of the 150 mm run (to make a mark on the run graph).
³The silicone adhesive was still attached to the probe at the maximum allowed travel distance of 150 mm. The low work of adhesion valve 0.30 indicates that the adhesive sample stretched easily with a low force.

Adhesive Samples 1, 3-8, and 10 had adhesion and elongation properties sufficient for adhering to mucocutaneous base and mucosal walls to seal around a stoma. Sample 2, which was formulated with 98.0 wt. % RTV-2 silicone composition and 2.0 wt. % cross linked polyacrylic acid polymer and polyacrylate based superabsorbent polymer, and Sample 9, which included 100 wt. % RTV-2 silicone composition, did not have adhesion and elongation properties sufficient for adhering to mucocutaneous base and mucosal walls to seal around a stoma.

### Moist Tissue Tack and Adhesion Test on TA-XT Plus Texture Analyzer

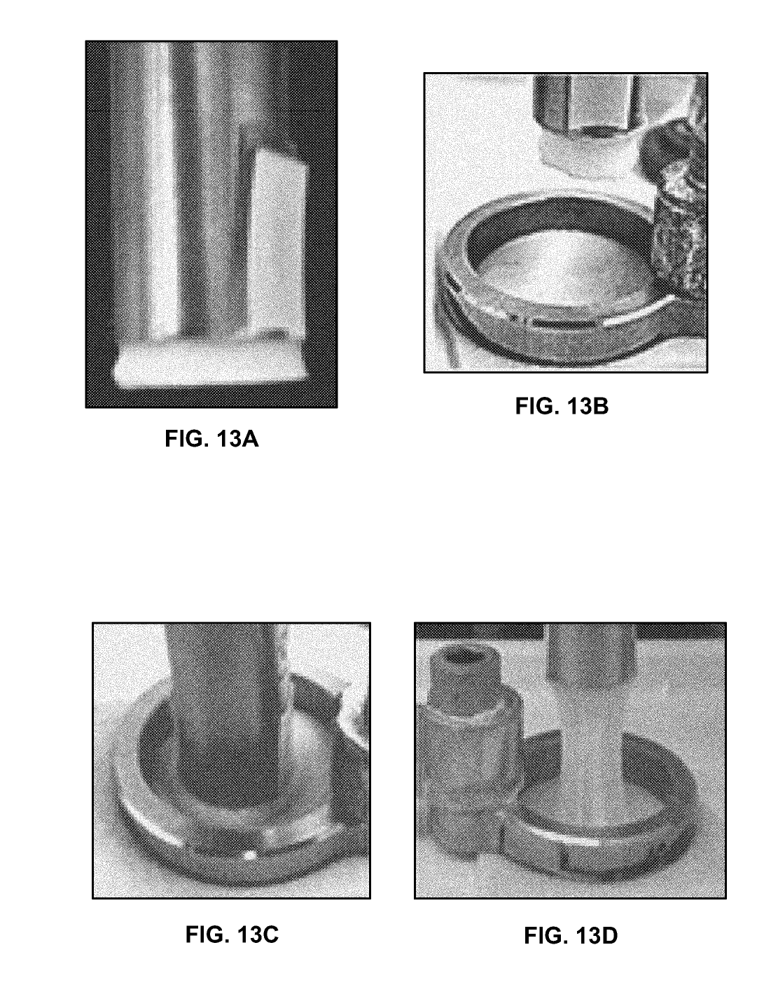

25 g of each of sample adhesive compositions for moist tissue was poured on a circular polycarbonate film having a thickness of 20 mil, while the film is held flat in a petri dish by a 4.50 inch diameter Aluminum ring. The polycarbonate circular film was pretreated with a primer. The adhesive samples were degassed for 5-10 min prior to curing in a conventional oven. Circular test specimens having a diameter of about 1 cm and a thickness of about 50 mil were cut out from the adhesive samples. Each of the test specimens was mounted on a flat stainless steel probe having a length of 2 inches and a diameter of 1 cm using a commercial double sided tape. The test specimens were allowed to stand at room temperature for minimum 24 hours prior to testing. FIG. 13A is a photograph of a test specimen mounted on the flat stainless steel probe.

A porcine epidermis having a thickness of 0.060±0.010 inches was cut to 1 inch×1 inch squares and conditioned in a 6.80 pH buffer (prepared according to USP 38-NF 33 (2015), page 7206-7207) for at least one hour prior to testing. For testing, TA-XT Plus texture analyzer load cell was first calibrated using a 2000 g weight. The flat stainless steel probe with an adhesive test specimen was attached to the TA-XT Plus probe arm. A 1 inch×1 inch moist porcine epidermis square was placed in a plastic tray with layers of paper towels. Three Kim-wipe tissue sheets in folded format were laid on the moist porcine epidermis square, and a 200 g Aluminum bar was gently laid on it for 15 seconds to absorb excess buffer solution. The moist porcine epidermis square was quickly transferred to a specimen table of the TA_XT2 Plus Texture Analyzer and held in place with a ring clamp. FIG. 13B is a photograph of a moist porcine epidermis square held in place with a ring clamp. FIG. 13C is a photograph of the flat stainless steel probe with an adhesive test specimen pressed again a moist porcine epidermis square. FIG. 13D is a photograph of the adhesive test specimen adhering to the moist porcine epidermis square and stretching during a test run. Test parameters included: test mode—tension, test speed—0.20 mm/sec, applied force—127 g, contact time—60 sec, trigger type—Auto, trigger force—5 g, post-test speed—0.20 mm/sec (a speed of the probe moving away from the porcine epidermis square after an adhesive test specimen was pressed against porcine epidermis square.) Test results are summarized in Table 2.

TABLE 2
Moist Tissue Tack and Adhesion Test Results

| | | | crosslinked polyacrylic | | Average Work | Average sample |
|---|---|---|---|---|---|---|
| | | RTV-2 | acid polymer + | | of Adhesion | elongation |
| | RTV-2 | silicone type | sodium polyacrylate | Average | (Area under | before detaching |
| Sample | silicone | (n - sample | based superabsorbent | Peak tack | the curve) | cleanly from the |
| # | (wt. %) | size) | polymer (wt. %) | (N) | (g · mm) | probe (mm) |
| 11 | 91 | Type 1 (n = 6) | 9.00 | 66.64 | 555.40 | 12.90 |
| 12 | 89.9 | Type 1 (n = 6) | 10.10 | 64.2 | 715.4 | 20.60 |
| 13¹ | 94.0 | Type 1 (n = 10) | 5.00 | 37.68 | 179.65 | 6.9 |
| 14 | 91.61 | Type 1 (n = 7) | 8.39 | 84.59 | 807.61 | 17.4 |
| 15 | 90.5 | Type 3 (n = 10) | 9.50 | 136.14 | 1299.85 | 15.2 |
| 16 | 90.5 | Type 1 (n = 10) | 9.50 | 127.97 | 1120.96 | 14.7 |

¹This run also included 1% Fibrillated HDPE fiber (5 μm diameter).

All of the adhesive Samples 11-16 detached cleanly from a moist porcine epidermis without leaving any residues or damaging the tissue, and exhibited adhesion and elongation properties against a moist tissue sufficient for adhering to mucocutaneous base and mucosal walls to seal around a stoma.

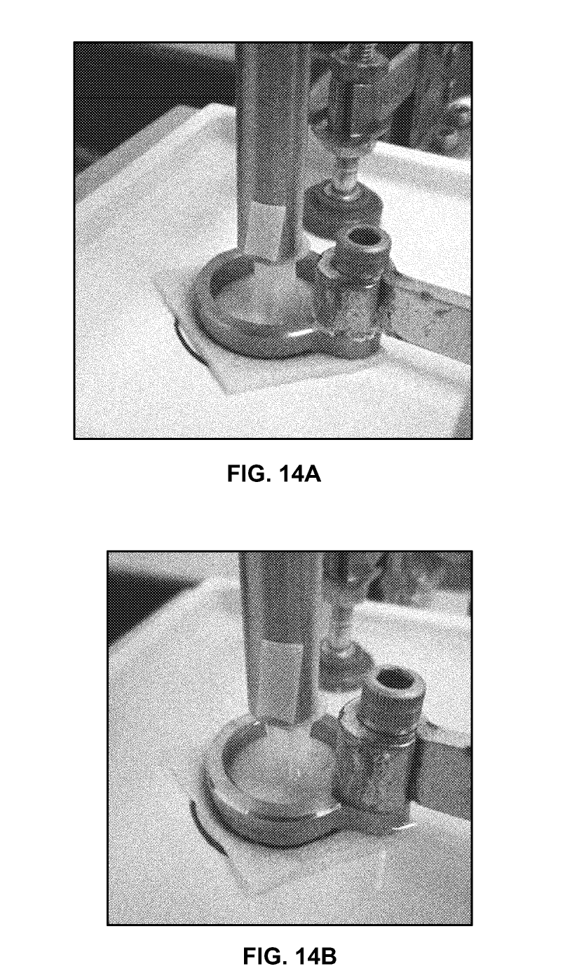

In a similar experiment, a test run was stopped after an adhesive test specimen formed from an adhesive composition comprising 90.5 wt. % of Type 3 RTV silicone and 9.5 wt. % of crosslinked polyacrylic acid polymer and sodium polyacrylate based superabsorbent polymer was attached to a moist porcine epidermis square and stretched during a moist tissue tack and adhesion test run, and a tray, in which the moist porcine square was held in place, was filled with pH 6.80 buffer solution overnight. FIG. 14A is a photograph showing the adhesive test specimen attached and stretched from a porcine epidermis immersed in buffer solution. The adhesive test specimen remained attached to the porcine epidermis in buffer solution for 16 hours after which the experiment was stopped. FIG. 14B is a photograph showing the adhesive test specimen attached and stretched from the porcine epidermis in buffer solution at 16th hour.

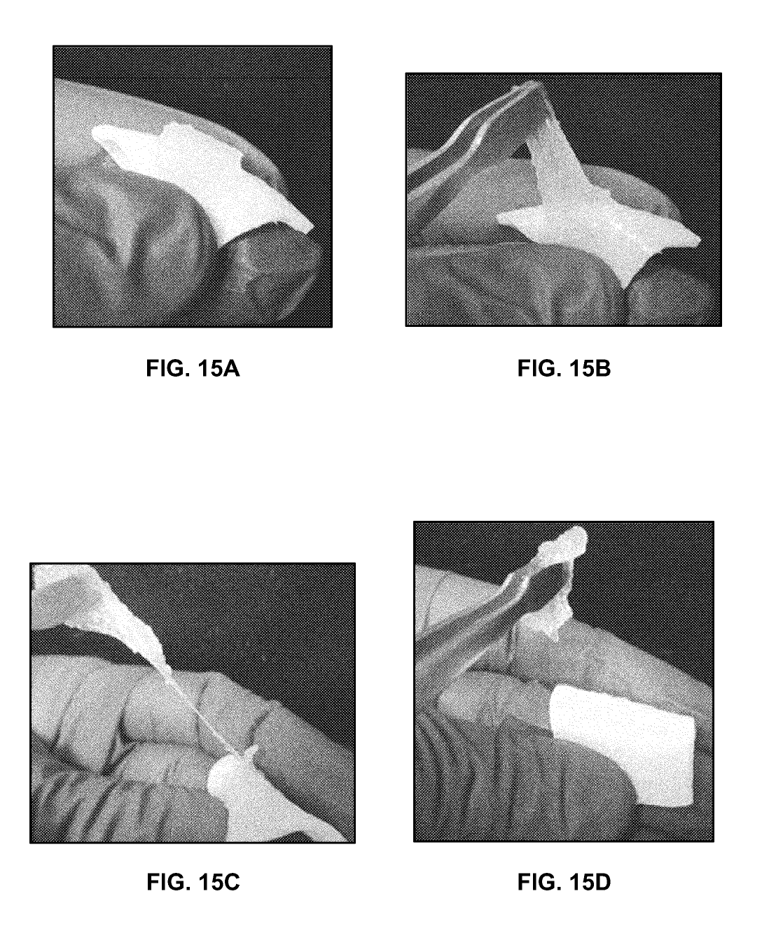

In another experiment, an adhesive sample formed from an adhesive composition comprising 90.61 wt. % of Type 1 RTV silicone and 9.39 wt. % of crosslinked polyacrylic acid polymer and sodium polyacrylate based superabsorbent polymer was attached to a moist porcine epidermis in a petri dish. The petri dish was filled with pH 6.8 buffer solution, and the adhesive sample and porcine epidermis were soaked at 25° C. for 5 days. The adhesive sample remained adhered to the moist porcine epidermis after 5 days, and detached cleanly from the moist porcine epidermis. FIGS. 15A-D are photographs showing the sample adhesive adhering to a moist porcine epidermis after soaking in pH 6.8 buffer at 25° C. for 5 days, and detaching cleanly from the moist porcine epidermis.

### Porcine Epidermis Peel Test on TA-XT Plus Texture Analyzer

10 g of each of sample adhesive compositions for moist tissue was poured into a 1 inch×6 inch plastic tray. The samples were degassed for 5-15 minutes to remove trapped air bubbles before curing. Cured adhesive samples were placed in a 25° C. chamber for conditioning overnight.

A porcine epidermis having a thickness of 0.060±0.010 inches was cut into 1 inch×6 inches strips and conditioned in 6.80 pH buffer solution (prepared according to USP 38-NF 33 (2015), page 7206-7207) for at least two hours prior to testing. One end of a porcine epidermis strip was stapled to a 1 inch×3 inches polycarbonate sheet having a thickness of 20 mil to act as a hanger for the TA-XT Plus grip clip. Just before testing, the moist porcine epidermis strip was removed from the buffer solution and placed in a tray containing layers of paper towels. Three Kim-wipe sheets were then placed on the porcine epidermis to cover its entire length, and a 1 Kg Steel bar, which also covered the entire length of the porcine epidermis, was placed on it for 15 seconds to remove loose buffer solution.

The moist porcine epidermis strip was then overlaid on a sample adhesive with the epidermis side facing the adhesive. A 20 mil thick sturdy plastic sheet was placed over the moist porcine epidermis, and a 2.28 Kg roller was gently rolled for 3 cycles over the plastic sheet to allow the moist porcine epidermis to come in full contact with the adhesive sample. A tray containing the laminated adhesive sample and moist porcine epidermis was then mounted on a 90° peel test platform, and the polycarbonate film, which was attached to the porcine epidermis, was latched tight using a foot pedal controller of the Texture Analyzer clamp. TA-XT Plus peel test parameters used: test speed—5 mm/sec, trigger force—5 g, trigger type—Auto, travel distance—150 mm. Test results are summarized in Table 3.

TABLE 3
Porcine Epidermis Peel Test Results

| | | RTV-2 | crosslinked polyacrylic | |
|---|---|---|---|---|
| | | silicone type | acid polymer + sodium | Average |
| | RTV-2 | or hydrocolloid | polyacrylate based | Peel |
| | silicone | type | superabsorbent polymer | Force |
| Sample # | (wt. %) | (n—sample size) | (wt. %) | (g) |
| 17 | 90.5 | Type 3 (n = 3) | 9.50 | 25.21 |
| 18 | 90.5 | Type 1 (n = 3) | 9.50 | 37.78 |
| 19 | 98.0 | Type 1 (n = 3) | 2.00 | 32.15 |
| 20 | 98.5 | Type 1 (n = 3) | 7.50 | 34.06 |
| 21 | 90.5 | Type 2 (n = 3) | 9.50 | 32.47 |
| 22 | 90.5 | Type 5 (n = 3) | 9.50 | 32.25 |
| 23¹ | N/A | Flextend ® (n = 3) | N/A | 14.20 |
| 24² | N/A | CeraPlus ® (n = 3) | N/A | 16.83 |

¹A 40 mil thick Flextend ® (1″ × 6″) attached to plastic tray with double sided tape
²A 40 mil thick CeraPlus ® (1′ × 6′) attached to plastic tray with double sided tape

Samples 17-22, which were adhesive compositions for moist tissue according to various embodiments, had significantly better peel force against a moist porcine epidermis when compared to known hydrocolloids (Samples 23 and 24), such as Flextend® and CeraPlus®, which are commercially available from Hollister.

Absorption Test

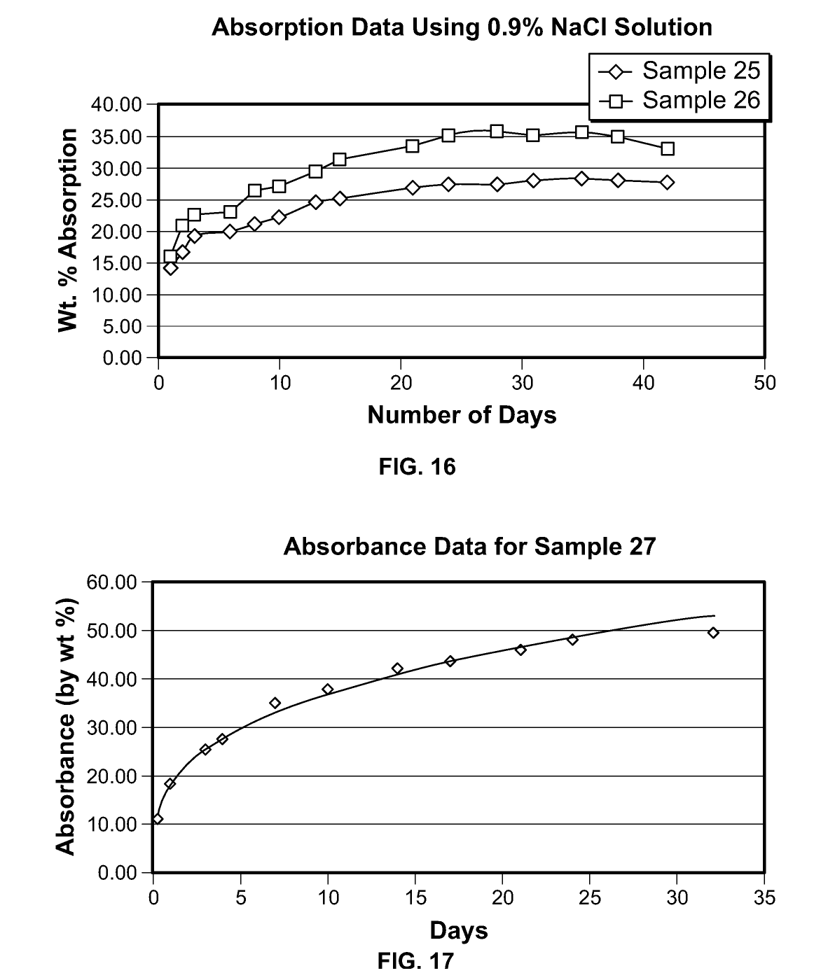

An adhesive composition comprising 90.5 wt. % of Type 3 RTV silicone and 9.5 wt. % of crosslinked polyacrylic acid polymer and sodium polyacrylate based superabsorbent polymer (Sample 25), and an adhesive composition comprising 90.5 wt. % of Type 1 RTV silicone and 9.5 wt. % of crosslinked polyacrylic acid polymer and sodium polyacrylate based superabsorbent polymer (Sample 26) were used to prepare test specimens. A sheet of each of the adhesive samples were formed to have a thickness of about 100 mil, and the adhesive sheet was punches into circular specimens having a diameter of 1 inch. Each of the adhesive specimens was weighed on a preweighed wire mesh and placed in a petri dish. 25 mL of 0.9% NaCl solution was added to the petri dish. 17 specimens of each of the sample adhesive compositions were prepared and tested. Each of the adhesive specimens was covered with a flange and a wire mesh to ensure that adhesive specimen was completely immersed under saline solution. At each data point, an adhesive specimen with a preweighed wire mesh was removed, gently dabbed with Kim Wipes to remove excess saline solution, and weighed on an analytical balance. Actual weight and hence the amount of saline solution absorbed was determined. FIG. 16 is a graph showing saline solution absorption rates of the adhesive samples.

None of the adhesive specimens fell apart or disintegrated after more than 40 days in saline solution. Further after soaking in saline solution for more than 40 days, adhesive samples still had significant adhesion to skin.

In a similar experiment, an adhesive composition comprising 90.5 wt. % of Type 1 RTV silicone and 9.5 wt. % of crosslinked polyacrylic acid polymer and sodium polyacrylate based superabsorbent polymer (Sample 27) was prepared into test specimens having a thickness of about 100 mil. The adhesive specimens were soaked in 0.9% NaCl solution, and weight gain of the adhesive specimens over time was plotted in a graph shown in FIG. 17. The adhesive specimens did not fall apart and retained the shape and structure after more than 30 days of soaking in 0.9% NaCl solution.

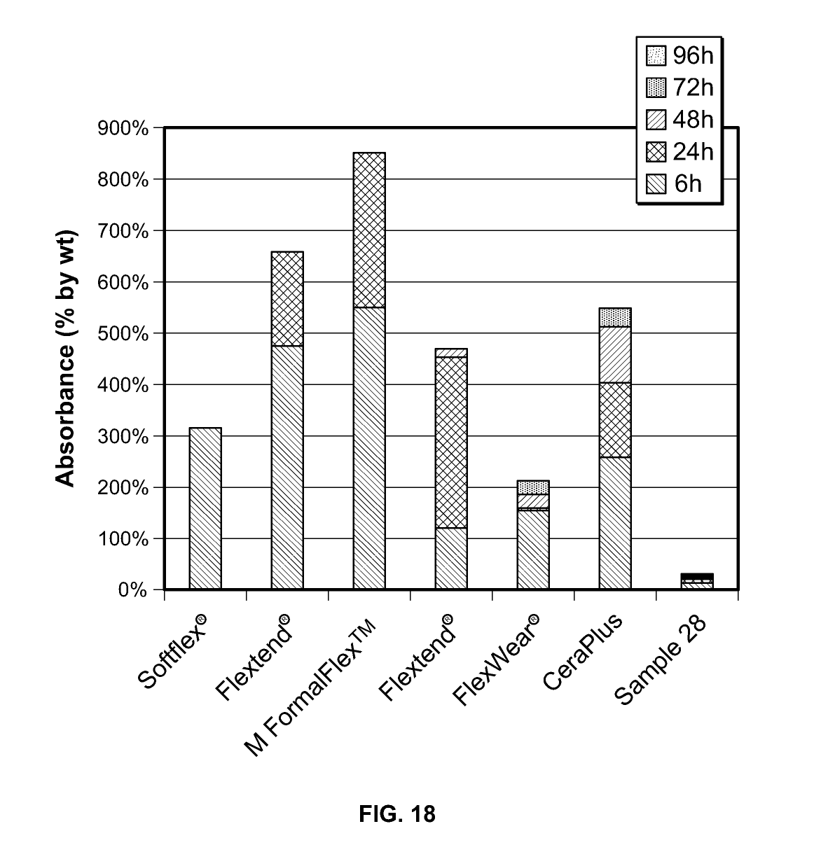

FIG. 18 is a bar graph showing absorbance of known hydrocolloids and an adhesive composition comprising 90.5 wt. % of Type 1 RTV silicone and 9.5 wt. % of crosslinked polyacrylic acid polymer and sodium polyacrylate based superabsorbent polymer (Sample 28) in 0.9% NaCl solution. As shown, the adhesive composition for moist tissue (Sample 28) had a significantly less absorbance when compared to the hydrocolloid samples (Softflex®, Flextend® M, FormaFlex™, Flextend®, FlexWear®, and CeraPlus®).

In a user test, a ring-like shape stoma seal was formed from an adhesive composition comprising 90.5 wt. % of Type 1 RTV silicone and 9.5 wt. % of crosslinked polyacrylic acid polymer and sodium polyacrylate based superabsorbent polymer, and the stoma seal was applied on a hydrocolloid skin barrier proximate an inlet opening. The hydrocolloid skin barrier with the stoma seal was attached to a user, such that user's stoma is received through a center opening stoma seal. The user wore the same stoma seal and hydrocolloid skin barrier for 72 hours, during which the user performed various physical activities, such as jogging, showering, etc. The user did not experience any leakage during use and liked the stoma seal as it provided added comfort and security. After 72 hours, the stoma seal on the user was evaluated by clinicians, which showed that the stoma seal formed a good seal around the base of the stoma.

All patents referred to herein, are hereby incorporated herein in their entirety, by reference, whether or not specifically indicated as such within the text of this disclosure.

In the present disclosure, the words “a” or “an” are to be taken to include both the singular and the plural. Conversely, any reference to plural items shall, where appropriate, include the singular.

From the foregoing it will be observed that numerous modifications and variations can be effectuated without departing from the true spirit and scope of the novel concepts of the present disclosure. It is to be understood that no limitation with respect to the specific embodiments illustrated is intended or should be inferred. The disclosure is intended to cover by the appended claims all such modifications as fall within the scope of the claims

1. An adhesive composition for moist tissue comprising:
about 80 wt. % to about 98 wt. % of a two-part addition curing silicone composition; and
about 2 wt. % to about 20 wt. % of at least one hydrophilic component;
wherein the adhesive composition is cured to form a viscoelastic adhesive that adheres to a moist mucocutaneous tissue and a mucosal tissue.

2. The adhesive composition of claim 1, wherein the at least one hydrophilic component comprises a crosslinked polyacrylic acid polymer.

3. The adhesive composition of claim 1, wherein the hydrophilic component comprises a sodium polyacrylate based superabsorbent polymer.

4. The adhesive composition of claim 1, wherein the two-part addition curing silicone composition comprises a component A including a platinum catalyst and vinyl functional polymers (R—CH═CH₂) and a component B comprising silicone hydride groups (—SiH).

5. The adhesive composition of claim 1, wherein the viscoelastic adhesive has a total work adhesion greater than about 7 N·mm and an elongation before detaching greater than about 3.0 mm and less than about 150 mm when tested according the Spherical Probe Tack and Adhesion test method described in Examples and Test Results section of the present disclosure.

6. The adhesive composition of claim 1, wherein the viscoelastic adhesive has a total work adhesion greater than about 100 g·mm and an elongation before detaching greater than about 5.0 mm and less than about 50 mm when tested according the Moist Tissue Tack and Adhesion test method described in Examples and Test Results section of the present disclosure.

7. The adhesive composition of claim 1, wherein the viscoelastic adhesive has saline solution absorption over 40 days of about 7 wt. % to about 80 wt. % when tested according to the Absorption Test in 0.9% NaCl solution described in Examples and Test Results section of the present disclosure.

8. The adhesive composition of claim 1, wherein the adhesive composition is formed into a stoma seal having a ring-like shape body, wherein the stoma seal maintains the shape and structure of the body after being soaked in a 0.9% NaCl solution for 40 days.

9. An ostomy skin barrier comprising:
a faceplate;
a first inlet opening defined in the faceplate;
a stoma seal provided in the first inlet opening, the stoma seal having a ring-like shape body including a second inlet opening configured to receive a stoma;
a first adhesive layer provided on the faceplate; and
a second adhesive layer provided on the faceplate surrounding the stoma seal;
wherein each of the stoma seal, the first adhesive, and the second adhesive is formed from a different adhesive formulation.

10. The ostomy skin barrier of claim 9, wherein the first adhesive layer is formed from a hydrophilic adhesive and the second adhesive layer is formed from a hydrophobic adhesive, wherein the second adhesive layer is arranged between the stoma seal and the first adhesive layer.

11. The ostomy skin barrier of claim 10, wherein the first adhesive layer is formed from a hydrocolloid adhesive or an acrylic adhesive, and the second adhesive layer is formed from a silicone adhesive.

12. The ostomy skin barrier of claim 9, wherein the stoma seal is formed from any of the adhesive composition of claim 1.

13. The ostomy skin barrier of claim 9, wherein the second adhesive layer having a ring-like shape is arranged on the first adhesive layer, such that an outer peripheral portion of the first adhesive layer remains exposed for attachment to a user, wherein the first inlet opening is defined by inner peripheries of the faceplate, the first adhesive layer, and the second adhesive layer, and the stoma seal is provided in the first opening, wherein the stoma seal has a thickness equal to or greater than a combined thickness of the faceplate, the first adhesive layer, and the second adhesive layer, such that the stoma seal extends longitudinally from a pouch side inner periphery of the faceplate to a body side inner periphery of the second adhesive layer, wherein a cover is provided on a body side surface of the stoma seal, and a sealing layer is provided on a pouch side surface of the stoma seal, wherein the pouch side surface of the stoma seal is secured to the sealing layer.

14. The ostomy skin barrier of claim 13, wherein a first release liner is provided on the exposed outer peripheral portion of the first adhesive layer, and a second release liner is provided on a body side surface the second adhesive layer, wherein the stoma seal has a thickness equal to or greater than a combined thickness of the faceplate, the first adhesive layer, the second adhesive layer, and the second release liner, such that the stoma seal extends longitudinally from a pouch side inner periphery of the faceplate to a body side inner periphery of the second release liner.

15. The ostomy skin barrier of claim 13, wherein the first adhesive is formed from an acrylic adhesive, and the second adhesive layer is formed from a hydrocolloid adhesive, and the sealing layer is formed from a silicone.

16. The ostomy skin barrier of claim 13, wherein the stoma seal is formed from the adhesive composition of claim 1.

17. The ostomy skin barrier of claim 16, wherein ostomy skin barrier further include a body side coupling ring attached on a pouch side surface of the faceplate, wherein the sealing layer is provided over the stoma seal and an inner peripheral portion of the faceplate inside an inner perimeter of the body side coupling ring.

18. An ostomy skin barrier comprising:
a faceplate including an opening defined by an inner periphery of the faceplate;
a first adhesive layer provided on a body side surface of the faceplate, wherein the first adhesive layer has an inner periphery that substantially lines up with the inner periphery of the faceplate;
a backing layer having a ring-like shape provided on an inner peripheral portion of the first adhesive, such that an outer peripheral portion of the first adhesive is exposed for attachment to a user, wherein an inner diameter of the backing layer is less than inner diameters of the faceplate and the first adhesive layer, such that an inner peripheral portion of the backing layer extends beyond the inner peripheries of the faceplate and the first adhesive layer;
a second adhesive layer having a ring-like shape provided on an outer peripheral portion of the backing layer, such that the outer peripheral portion of the backing layer is secured between the first adhesive layer and the second adhesive layer, wherein the second adhesive layer includes an opening defined by an inner periphery of the second adhesive; and
a stoma seal provided in the opening of the second adhesive layer and attached to an inner peripheral portion of the backing layer, the stoma seal having a ring-like shape body including an inlet opening configured to receive a stoma.

19. The ostomy skin barrier of claim 18, wherein each of the stoma seal, the first adhesive layer, and the second adhesive layer is formed from a different adhesive formulation.

20. The ostomy skin barrier of claim 18, wherein the first adhesive layer is formed from an acrylic adhesive, and the second adhesive layer is formed from a hydrocolloid adhesive, and the backing layer is formed from a polymeric film having a thickness of about 1 mil to about 7 mil.

21. The ostomy skin barrier of claim 18, wherein the stoma seal is formed from any of the adhesive composition of claim 1, and the backing layer is formed from a thermoplastic urethane-phenoxy film having a thickness of about 5 mil.

22. The ostomy skin barrier of claim 18, wherein a first release liner is provided on the exposed outer peripheral portion of the first adhesive layer, and a second release liner is provided on a body side surface of the second adhesive layer, and a cover is provided on a body side surface of the stoma seal.
